(19) United States
(12) Patent Application Publication
(10) Pub. No.: US 2026/0096742 A1
(43) Pub. Date: Apr. 9, 2026

(54) RESPIRATION RATE ESTIMATION SYSTEM, RESPIRATION RATE ESTIMATION DEVICE, RESPIRATION RATE ESTIMATION METHOD, AND RESPIRATION RATE ESTIMATION PROGRAM

(71) Applicant: PANASONIC INTELLECTUAL PROPERTY MANAGEMENT CO., LTD., Osaka (JP)

(72) Inventors: Takeo UETA, Kanagawa (JP); Makoto YASUGI, Tokyo (JP); Hiroshi NOGUCHI, Kanagawa (JP); Toru OKADA, Kanagawa (JP); Kosuke ONO, Kanagawa (JP)

(73) Assignee: PANASONIC INTELLECTUAL PROPERTY MANAGEMENT CO., LTD., Osaka (JP)

(21) Appl. No.: 19/110,744

(22) PCT Filed: Aug. 10, 2023
(86) PCT No.: PCT/JP2023/029283

§ 371 Date: Nov. 28, 2025

(30) Foreign Application Priority Data
Sep. 12, 2022 (JP) 2022-144863

(51) Int. Cl. A61B 5/08; A61B 5/00; A61B 5/05; G01S 13/89

(52) U.S. Cl. CPC A61B 5/0816; A61B 5/0022; A61B 5/05; A61B 5/0806; A61B 5/7275; G01S 13/89

(57) ABSTRACT

A breathing rate estimation system includes at least one radar type sensor installed in a monitoring area, an information processing device configured to execute person detection processing of acquiring sensor data output from the sensor and detecting a person present in the monitoring area using the acquired sensor data and breathing rate estimation processing of estimating a breathing rate of the person, and a display device configured to display the breathing rate of the person estimated in the breathing rate estimation processing. The information processing device determines whether the person is in a specific state based on a detection result of the person by the person detection processing, and executes the breathing rate estimation processing when it is determined that the person is in the specific state.

### TECHNICAL FIELD

The present disclosure relates to a breathing rate estimation system, a breathing rate estimation device, a breathing rate estimation method, and a breathing rate estimation program.

### BACKGROUND ART

A technique for estimating a breathing rate of a person using radio waves is known. Patent Literature 1 discloses a respiration detection system including a transmission unit that transmits radio waves to a plurality of positions including a position where a subject is present, a reception unit that receives reflected waves obtained by reflecting the radio waves, a phase fluctuation calculation unit that calculates a phase fluctuation of the reflected waves, a generation unit that performs a Fourier transform on the phase fluctuation and generates a spectrogram indicating a relationship between a time at which the reflected waves are received and a frequency component included in the reflected waves, and a breathing rate estimation unit that estimates a breathing rate of the subject by outputting a probability that the subject takes breaths at a predetermined frequency for each frequency based on the spectrogram and calculating a weighted average of the frequency by using the probability as a weight.

### CITATION LIST

### Patent Literature

Patent Literature 1: WO2021/039601
Patent Literature 2: JP6900390B

### SUMMARY OF INVENTION

### Technical Problem

However, there is a case where the person is in a situation inappropriate for estimation of the breathing rate by radio waves, such as a case where the person is actively acting. The breathing rate estimated in such a situation is an erroneous breathing rate different from an original breathing rate, which leads to a decrease in reliability of a system that estimates the breathing rate using radio waves.

An object of the present disclosure is to provide a technique for preventing an erroneous breathing rate from being output by appropriately estimating a breathing rate according to a situation of a person in a system that estimates the breathing rate of the person using radio waves.

### Solution to Problem

A breathing rate estimation system according to an aspect of the present disclosure includes at least one radar type sensor installed in a monitoring area, an information processing device configured to execute person detection processing of acquiring sensor data output from the sensor and detecting a person present in the monitoring area using the acquired sensor data and breathing rate estimation processing of estimating a breathing rate of the person, and a display device configured to display the breathing rate of the person estimated in the breathing rate estimation processing, in which the information processing device determines whether the person is in a specific state based on a detection result of the person by the person detection processing, and executes the breathing rate estimation processing when it is determined that the person is in the specific state.

A breathing rate estimation device according to an aspect of the present disclosure is a breathing rate estimation device including a processor and a memory, in which the processor cooperates with the memory to acquire sensor data output from at least one radar type sensor installed in a monitoring area, execute person detection processing of detecting a person present in the monitoring area using the sensor data, determine whether the person is in a specific state based on a detection result of the person in the person detection processing, execute breathing rate estimation processing of estimating a breathing rate of the person using the sensor data when it is determined that the person is in the specific state, and output the breathing rate of the person estimated in the breathing rate estimation processing.

A breathing rate estimation method according to an aspect of the present disclosure includes acquiring sensor data output from at least one radar type sensor installed in a monitoring area, executing person detection processing of detecting a person present in the monitoring area using the sensor data, determining whether the person is in a specific state based on a detection result of the person in the person detection processing, executing breathing rate estimation processing of estimating a breathing rate of the person using the sensor data when it is determined that the person is in the specific state, and outputting the breathing rate of the person estimated in the breathing rate estimation processing.

A breathing rate estimation program according to an aspect of the present disclosure causes a computer to execute processing, the processing including acquiring sensor data output from at least one radar type sensor installed in a monitoring area, executing person detection processing of detecting a person present in the monitoring area using the sensor data, determining whether the person is in a specific state based on a detection result of the person in the person detection processing, executing breathing rate estimation processing of estimating a breathing rate of the person using the sensor data when it is determined that the person is in the specific state, and outputting the breathing rate of the person estimated in the breathing rate estimation processing.

These comprehensive or specific aspects may be implemented by a system, a device, a method, an integrated circuit, a computer program, or a recording medium, or any combination of the system, the device, the method, the integrated circuit, the computer program, and the recording medium.

### Advantageous Effects of Invention

According to the present disclosure, it is possible to prevent an erroneous breathing rate from being output by appropriately estimating a breathing rate according to a situation of a person.

### BRIEF DESCRIPTION OF DRAWINGS

FIG. 1 is a diagram illustrating a configuration example of a breathing rate estimation system according to an embodiment;

FIG. 2 is a diagram illustrating a configuration example of a radar device according to the present embodiment:

FIG. 3 is a block diagram illustrating a configuration example of a breathing rate estimation device according to the present embodiment:

FIG. 4 is a block diagram illustrating a configuration example of a person detection processing unit according to the present embodiment:

FIG. 5 is a block diagram illustrating a configuration example of a breathing rate estimation processing unit according to the present embodiment:

FIG. 6 is a flowchart illustrating a processing example performed by the breathing rate estimation system according to the present embodiment:

FIG. 7 is a flowchart illustrating the processing example continued from FIG. 6: FIG. 8 is a diagram illustrating a method for determining whether a person is in a static state according to the present embodiment:

FIGS. 9A and 9B are a diagram illustrating correction of IQ data according to the present embodiment:

FIG. 10 is an image diagram of a waveform buffer according to present embodiment: FIG. 11 is a diagram illustrating an example of a phase waveform spectrum according to the present embodiment:

FIG. 12 is a diagram illustrating a display example of a breathing rate and a likelihood according to the present embodiment:

FIG. 13 is a diagram illustrating an example of a reflection position of a radar with respect to a body surface of a person according to the present embodiment;

FIGS. 14A, 14B and 14C are graphs illustrating an example of phase waveforms and phase waveform spectra for the abdomen, the chest, and the leg according to the present embodiment:

FIG. 15 is a diagram illustrating a method for calculating the likelihood according to the present embodiment;

FIGS. 16A, 16B and 16C are diagrams illustrating an example of the phase waveform spectra and likelihoods for the abdomen, the chest, and the leg according to the present embodiment; and

FIG. 17 is a diagram illustrating a hardware configuration example of an information processing device (computer) according to the present disclosure.

### DESCRIPTION OF EMBODIMENTS

Hereinafter, an embodiment of the present disclosure will be described in detail with reference to the drawings as appropriate. However, unnecessarily detailed description may be omitted. For example, detailed description of already well-known matters and redundant description of substantially the same configuration may be omitted. This is to avoid unnecessary redundancy of the following description and to facilitate understanding of those skilled in the art. The accompanying drawings and the following description are provided for those skilled in the art to fully understand the present disclosure, and are not intended to limit the subject matter described in the claims.

### Present Embodiment

### <Outline>

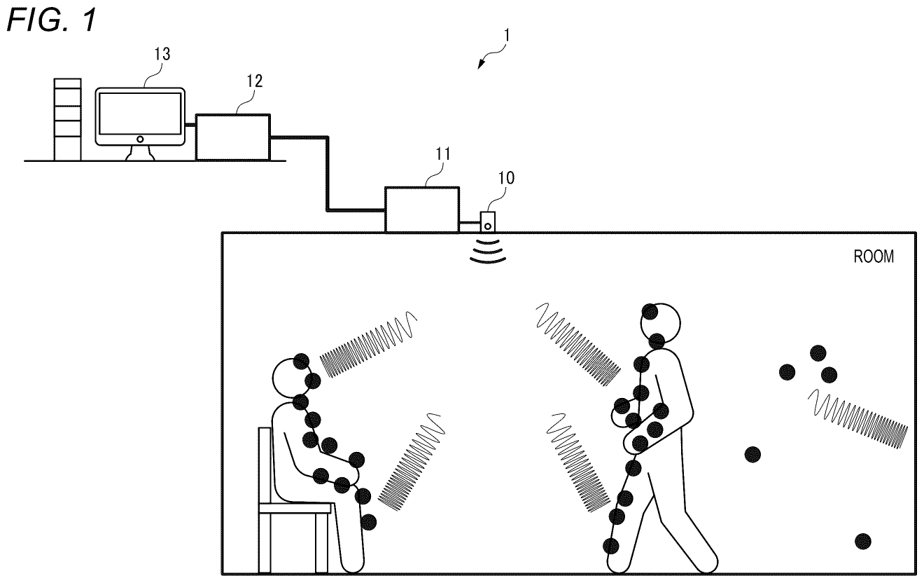

FIG. 1 is a diagram illustrating a configuration example of a breathing rate estimation system 1 according to an embodiment.

The breathing rate estimation system I includes a radar device 10, a breathing rate estimation device 11, a browsing processing device 12, and a display device 13. The radar device 10 is connected to the breathing rate estimation device 11 through a predetermined electrical cable. The browsing processing device 12 can transmit and receive data to and from the breathing rate estimation device 11 through a communication network such as a wired local area network (LAN) or a wireless LAN. The display device 13 is connected to the browsing processing device 12 through a predetermined electrical cable.

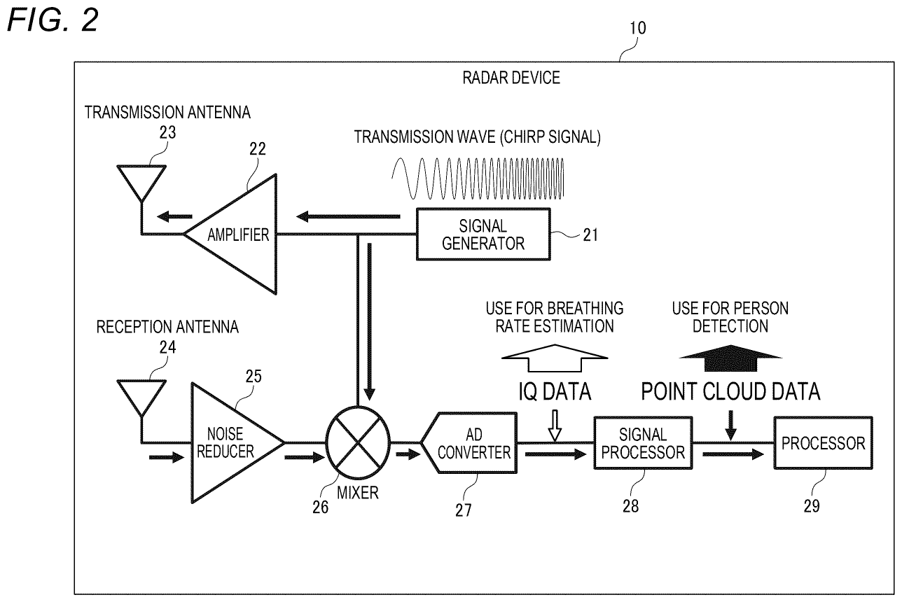

The radar device 10 is an example of a radar type sensor. The radar device 10 is installed on the ceiling of a room, which is an example of a monitoring area. However, an installation position of the radar device 10 is not limited to the ceiling, and the radar device 10 may be installed at an appropriate position in accordance with the shape of the room or the like. The radar device 10 transmits transmission waves (radar) in a millimeter wave band (for example, a wavelength of 1 cm to 1 mm) from a transmission antenna 23 (see FIG. 2). The transmission waves are reflected by a body surface of each person present in the room. The radar device 10 receives the reflected waves reflected by the body surface of the person by a reception antenna 24 (see FIG. 2). The wavelength used for the transmission waves (radar) is not limited to the millimeter wave band, and may be a wavelength longer than the millimeter wave band or a wavelength shorter than the millimeter wave band. Since the body surface fluctuates due to the breathing of the person, a phase of the reflected waves also changes in accordance with the fluctuation of the body surface. Therefore, by detecting the change in the phase of the reflected waves, the fluctuation of the body surface of the person, that is, the breathing rate of the person can be estimated.

The breathing rate estimation device 11 estimates the breathing rate of each person present in the room based on sensor data regarding the reflected waves transmitted from the radar device 10, and transmits information on the estimated breathing rate of each person (hereinafter, referred to as breathing rate information) to the browsing processing device 12.

The browsing processing device 12 receives the breathing rate information from the breathing rate estimation device 11, and displays an image, characters, and the like indicating the breathing rate and the like of each person present in the room on the display device 13 based on the breathing rate information. A user can check the breathing rate and the like of each person present in the room by viewing information displayed on the display device 13.

A system that estimates the breathing rate of a person using a radar can estimate the breathing rate of a person in a non-contact manner while taking privacy into consideration. In addition, this system can collectively estimate the breathing rates of a plurality of persons present in the room. In addition, this system can estimate the breathing rate of the person even when the inside of the room is dark, such as when the person is sleeping.

However, there is a case where a person is in a situation inappropriate for estimation of a breathing rate by transmission waves (radar), such as a case where the person is actively acting. In addition, since the transmission waves are reflected at various positions on the body surface of the person, the received reflected waves also include reflected waves from a position on the body surface inappropriate for estimation of the breathing rate. In a case where the breathing rate is estimated using reflected waves in a situation inappropriate for estimation of the breathing rate, and/or in a case where the breathing rate is estimated using the reflected waves from a position of the body surface inappropriate for estimation of the breathing rate, an erroneous breathing rate greatly different from an original correct breathing rate is output. This leads to a decrease in reliability of the system that estimates respiration of a person using a radar.

Therefore, the following describes the breathing rate estimation system 1 that selects reflected waves appropriate for estimation of the breathing rate to prevent output of an erroneous breathing rate significantly different from the original correct breathing rate.

### <Configuration of Radar Device>

FIG. 2 is a diagram illustrating a configuration example of the radar device 10 according to the present embodiment.

The radar device 10 includes a signal generator 21, an amplifier 22, a transmission antenna 23, the reception antenna 24, a noise reducer 25, a mixer 26, an AD converter 27, a signal processor 28, and a processor 29. A plurality of transmission antennas 23 and a plurality of reception antennas 24 may be provided. That is, the radar device 10 may include an array antenna including a plurality of transmission antennas 23 and a plurality of reception antennas 24.

The signal generator 21 generates and outputs transmission waves. The transmission waves may be a chirp signal whose frequency changes with time in a predetermined cycle. That is, the signal generator 21 may generate transmission waves in a frequency modulated continuous wave (FMCW) format.

The amplifier 22 amplifies the transmission waves output from the signal generator 21.

The transmission antenna 23 transmits the transmission waves output from the amplifier 22 to a space.

The reception antenna 24 receives the reflected waves that arrive when the transmission waves are reflected by the body surface of the person.

The noise reducer 25 reduces noise of the reflected waves received by the reception antenna 24 and outputs the reflected waves in which the noise is reduced.

The mixer 26 generates and outputs a differential signal between the transmission waves output from the signal generator 21 and reception waves output from the noise reducer 25.

The AD converter 27 converts the analog differential signal output from the mixer 26 into a digital differential signal and outputs the digital differential signal.

The signal processor 28 generates point cloud data based on the differential signal output from the AD converter 27. For example, the signal processor 28 performs Range fast Fourier transform (FFT) on the differential signal and calculates a distance to a reflection point. For example, the signal processor 28 performs arrival direction estimation on the differential signal and calculates an angle of the direction of the reflection point. For example, the signal processor 28 performs Doppler FFT on the differential signal and calculates the Doppler velocity of the reflection point. Then, the signal processor 28 generates the point cloud data by associating the distance, the angle, and the Doppler velocity with each reflection point. That is, the point cloud data is a set of reflection points.

The processor 29 transmits the differential signal as in-phase/quadrature-phase (IQ) data to the breathing rate estimation device 11, and transmits the point cloud data to the breathing rate estimation device 11.

### <Configuration of Breathing Rate Estimation Device>

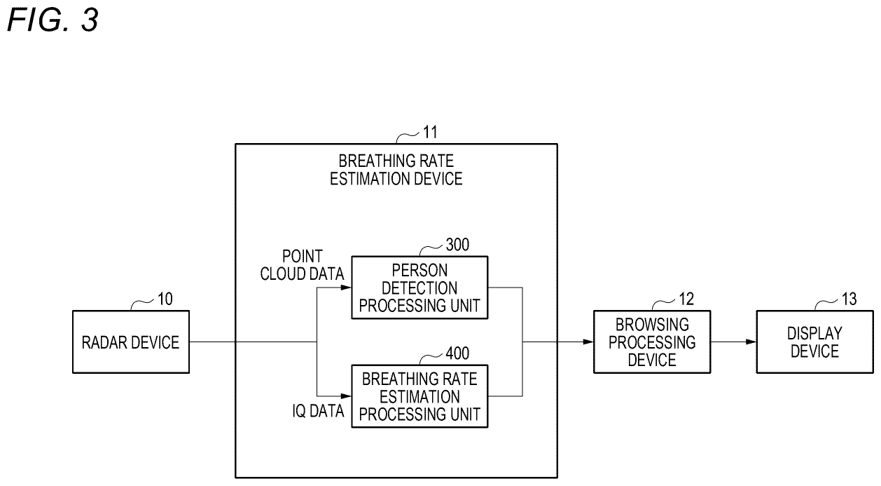

FIG. 3 is a block diagram illustrating a configuration example of the breathing rate estimation device 11 according to the present embodiment.

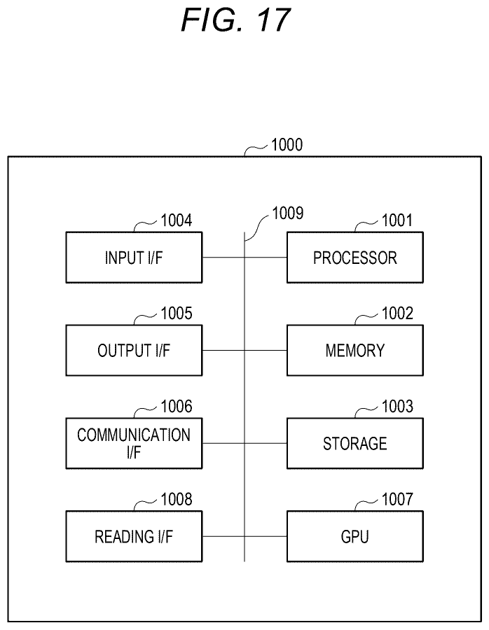

The breathing rate estimation device 11 includes a person detection processing unit 300 and a breathing rate estimation processing unit 400. The breathing rate estimation device 11 may include a processor 1001 and a memory 1002 as illustrated in FIG. 17 to be described later, and the functions of the person detection processing unit 300 and the breathing rate estimation processing unit 400 may be realized by the processor 1001 executing a computer program in cooperation with the memory 1002 and the like.

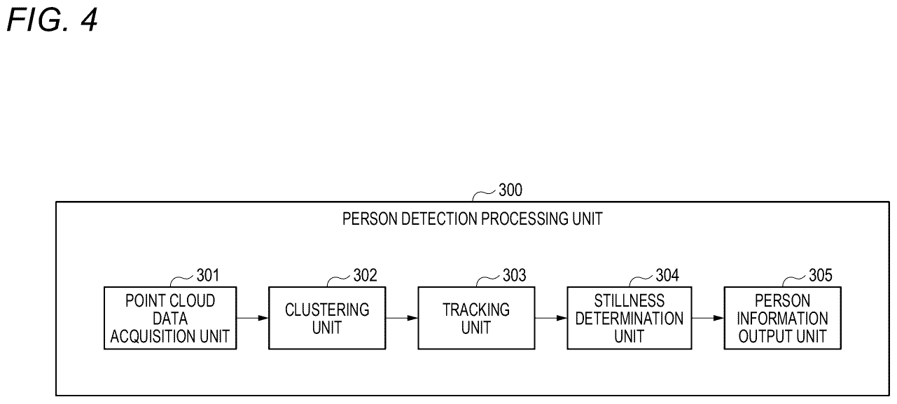

The person detection processing unit 300 detects the number of persons present in the room, a position of each person, and a static state of each person based on the point cloud data received from the radar device 10. Details of the person detection processing unit 300 will be described later (see FIGS. 4, 6, and 7).

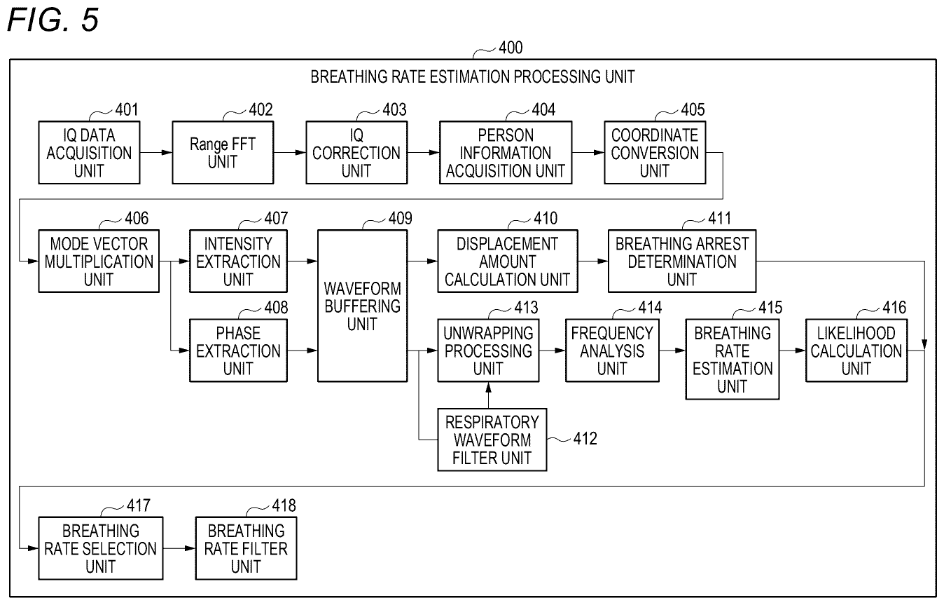

The breathing rate estimation processing unit 400 estimates the breathing rate of the person in a specific state among the persons detected by the person detection processing unit 300 based on the IQ data received from the radar device 10. The specific state is, for example, a state in which a person is substantially still. In this way, by estimating the breathing rate for a person in the static state and not estimating the breathing rate for a person who is not in the static state, it is possible to prevent the breathing rate estimation processing unit 400 from outputting an erroneous breathing rate. Further, since the breathing rate is not estimated for a person who is not in the static state, the breathing rate estimation processing unit 400 does not need to execute unnecessary processing of estimating an erroneous breathing rate. Details of the breathing rate estimation processing unit 400 will be described later (see FIGS. 5, 6, and 7).

The browsing processing device 12 causes the display device 13 to display the number of persons, the position of each person, and information indicating whether each person is in the static state detected by the person detection processing unit 300, and the breathing rate of the person, a likelihood indicating the certainty of the breathing rate, and the like estimated by the breathing rate estimation processing unit 400. The likelihood will be described in detail later. However, the browsing processing device 12 does not necessarily display all of these pieces of information on the display device 13, and may display at least one of these pieces of information on the display device 13.

### <Breathing Rate Estimation Method>

FIG. 4 is a block diagram illustrating a configuration example of the person detection processing unit 300 according to the present embodiment.

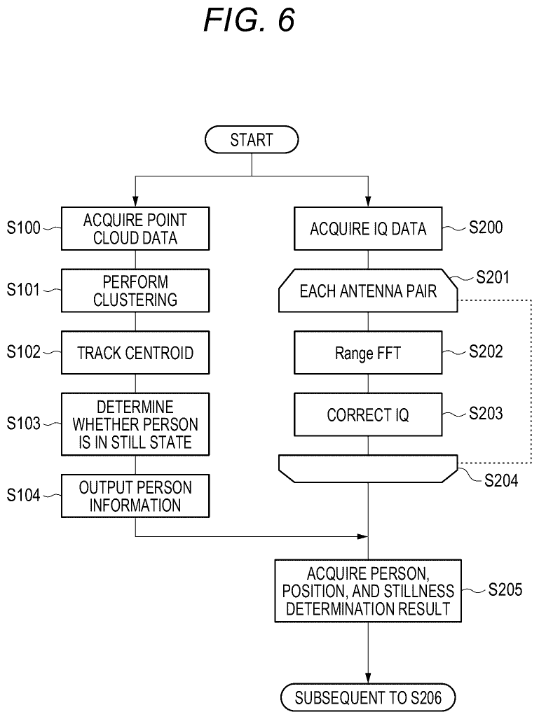

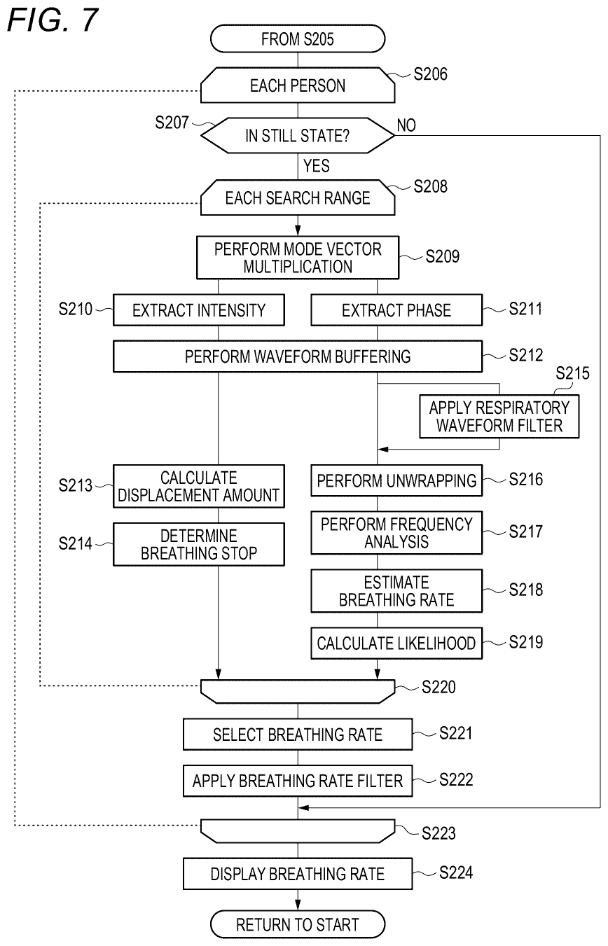

As illustrated in FIG. 4, the person detection processing unit 300 includes a point cloud data acquisition unit 301, a clustering unit 302, a tracking unit 303, a stillness determination unit 304, and a person information output unit 305. Details of processing performed by these components will be described with reference to flowcharts illustrated in FIGS. 6 and 7 to be described later.

FIG. 5 is a block diagram illustrating a configuration example of the breathing rate estimation processing unit 400 according to the present embodiment.

As illustrated in FIG. 5, the breathing rate estimation processing unit 400 includes an IQ data acquisition unit 401, a Range FFT unit 402, an IQ correction unit 403, a person information acquisition unit 404, a coordinate conversion unit 405, a mode vector multiplication unit 406, an intensity extraction unit 407, a phase extraction unit 408, a waveform buffering unit 409, a displacement amount calculation unit 410, a breathing arrest determination unit 411, a respiratory waveform filter unit 412, an unwrapping processing unit 413, a frequency analysis unit 414, a breathing rate estimation unit 415, a likelihood calculation unit 416, a breathing rate selection unit 417, and a breathing rate filter unit 418. Details of processing performed by these components will be described with reference to the flowcharts illustrated in FIGS. 6 and 7 to be described later.

FIG. 6 is a flowchart illustrating a processing example performed by the breathing rate estimation system 1 according to the present embodiment. FIG. 7 is a flowchart illustrating a processing example continued from FIG. 6. Next, processing performed by the breathing rate estimation system I will be described with reference to FIGS. 4, 5, 6, and 7.

The person detection processing unit 300 performs the following processing from step S100 to step S104.

(S100) The point cloud data acquisition unit 301 acquires point cloud data from the radar device 10.

(S101) The clustering unit 302 performs clustering on the point cloud data acquired in step S100 while removing noise, and generates at least one cluster. For example, the clustering unit 302 may remove multipath noise based on a difference in density using a density-based spatial clustering of applications with noise (DBSCAN) technique. One cluster corresponds to point cloud data of reflected waves from one person.

(S102) The tracking unit 303 tracks a centroid of each cluster generated in step S101. The centroid of the cluster corresponds to the position of one person, and one tracking corresponds to a movement path of one person. Note that the tracking unit 303 may delete a cluster for which tracking fails. Accordingly, a cluster that does not correspond to a person can be deleted, and the reliability of the cluster is improved. The tracking unit 303 may assign a target ID to a cluster for which tracking is successful. Accordingly, the target ID for distinguishing the person is assigned to the cluster corresponding to each person present in the room.

(S103) The stillness determination unit 304 determines whether the person is in a static state based on the tracking result in step S102. Next, a method for determining whether a person is in the static state will be described with reference to FIG. 8.

FIG. 8 is a diagram illustrating the method for determining whether a person is in the static state according to the present embodiment.

The stillness determination unit 304 has a first speed threshold value Vₜₕ₁ and a second speed threshold value Vₜₕ₂. Vₜₕ₂ is larger than Vₜₕ₁. Here, a speed section of 0 or more and Vₜₕ₁ or less is referred to as a first speed section, a speed section of larger than Vₜₕ₁ and Vₜₕ₂ or less is referred to as a second speed section, and a speed section of larger than Vₜₕ₂ is referred to as a third speed section.

The stillness determination unit 304 calculates a person speed v based on the tracking result of the person by the tracking unit 303.

The stillness determination unit 304 includes a counter having an initial value of 0 and a predetermined upper limit value. Further, the stillness determination unit 304 has a predetermined stillness determination threshold value set between 0 and the upper limit value. The stillness determination unit 304 determines that the person is in the static state when the counter is larger than the stillness determination threshold value and equal to or smaller than the upper limit value, and determines that the person is not in the static state when the counter is equal to or larger than 0) and equal to or smaller than the stillness determination threshold value.

The stillness determination unit 304 counts up the counter while the person speed v is within the first speed section. The stillness determination unit 304 counts down the counter while the person speed v is within the second speed section. When the person speed v is within the third speed section, the stillness determination unit 304 counts down the counter while the counter is larger than the stillness determination threshold value, and resets the counter (that is, the initial value 0) when the counter is equal to or smaller than the stillness determination threshold value.

Accordingly, the stillness determination unit 304 can prevent unstable determination as to whether the person is in the static state with a small movement. That is, the stillness determination unit 304 can stably determine whether the person is in the static state by a method illustrated in FIG. 8. In addition, the stillness determination unit 304 can quickly determine that the person is not in the static state when the person moves greatly by the method illustrated in FIG. 8.

(S104) The person information output unit 305 outputs the person information including the target ID based on the identification of the cluster, the person position based on the centroid of the cluster, the person speed based on the tracking result, and the stillness determination result to the breathing rate estimation processing unit 400.

The breathing rate estimation processing unit 400 performs the following processing from step S200 to step S224.

(S200) The IQ data acquisition unit 401 acquires the IQ data from the radar device 10.

(S201) The breathing rate estimation processing unit 400 selects an unselected one of a plurality of antenna pairs (that is, a plurality of virtual antennas) which are pairs of the transmission antenna 23 and the reception antenna 24, and performs the processing of step S201 to step S204 on the IQ data of the selected antenna pair.

(S202) The Range FFT unit 402 performs FFT on the IQ data to generate spectrum data indicating the cumulative time of the spectrum power for each range bin.

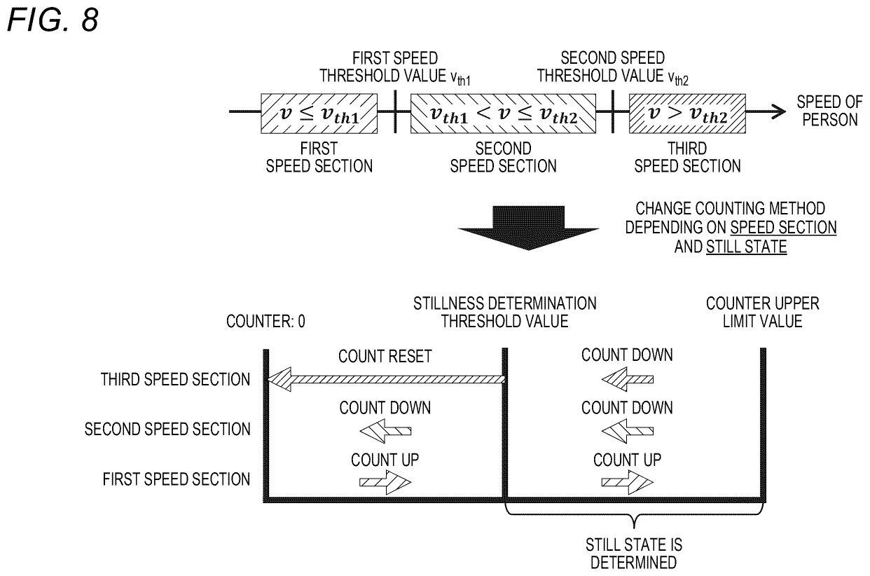

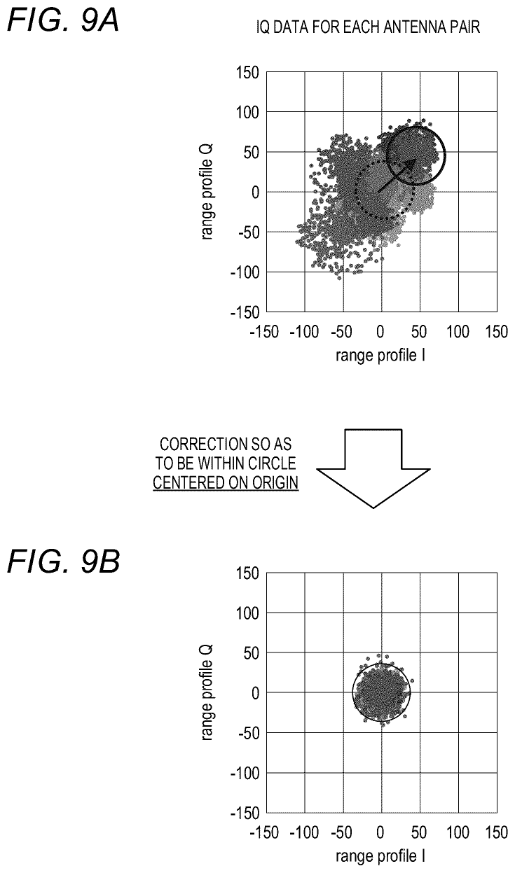

(S203) The IQ correction unit 403 corrects the IQ data as illustrated in FIG. 9A extracted in step S202 so that the IQ data falls within a circle centered on an origin on the complex plane as illustrated in FIG. 9B, as illustrated in the diagram illustrating the correction of the IQ data in FIGS. 9A and 9B. In the graph illustrated in FIGS. 9A and 9B, a horizontal axis represents the I component of the IQ data, and a vertical axis represents the Q component of the IQ data. As a result, the deviation of the reflection intensity on the complex plane is corrected, and the original phase component can be calculated in the subsequent processing.

(S204) After performing the processing from step S201 to step S204 for the IQ data of all the antenna pairs, the breathing rate estimation processing unit 400 advances the processing to the next step S205.

(S205) The person information acquisition unit 404 acquires the person information output from the person information output unit 305 of the person detection processing unit 300. That is, the person information acquisition unit 404 acquires the target ID, the position (distance and angle (elevation angle and azimuth angle)), and the stillness determination result of each person present in the room, which are obtained from the point cloud data.

Then, the breathing rate estimation processing unit 400 advances the processing to step S206 illustrated in FIG. 7.

(S206) The breathing rate estimation processing unit 400 specifies one or a plurality of persons present in the room based on the target ID of the person information acquired in step S205, and selects one unselected person among the specified one or plurality of persons. Then, the breathing rate estimation processing unit 400 performs the processing from step S206 to step S223 for the selected person.

(S207) The breathing rate estimation processing unit 400 determines whether the person is in the static state based on the stillness determination result of the person information acquired in step S205.

(S207: NO) When it is determined that the person is not in the static state, the breathing rate estimation processing unit 400 advances the processing to step S223. This is because there is a high possibility that an erroneous breathing rate is estimated when the person is not in the static state.

(S207: YES) When it is determined that the person is in the static state, the breathing rate estimation processing unit 400 advances the processing to the next step S208.

(S208) The breathing rate estimation processing unit 400 creates a search range based on the person position detected by the person detection processing unit 300, and performs the processing of step S208 to step S220 for each position of the search range. For example, when the distance of the detected person position is 2 m, the elevation angle is 30 degrees, and the azimuth angle is 40 degrees, the distance of the search range may be 1 m to 3 m, the elevation angle may be 25 degrees to 35 degrees, and the azimuth angle may be 35 degrees to 45 degrees. (S209) The mode vector multiplication unit 406 performs mode vector multiplication

on the IQ data of the designated range bin of the search range, and extracts the IQ data in the arrival direction. That is, the mode vector multiplication unit 406 strengthens the signal of the reflected waves obtained from the detection position of the person. The following processing of step S210 to step S219 are performed using the extracted IQ data.

(S210) The intensity extraction unit 407 extracts an intensity (amplitude) of the IQ data extracted in step S209.

(S211) The phase extraction unit 408 extracts the phase of the IQ data extracted in step S209.

(S212) The waveform buffering unit 409 buffers the intensity extracted in step S210 as needed. Accordingly, an intensity waveform indicating a temporal change in intensity is buffered in the waveform buffering unit 409. The waveform buffering unit 409 buffers the phase extracted in step S211 as needed. Accordingly, the phase waveform indicating a temporal change in the phase is buffered in the waveform buffering unit 409.

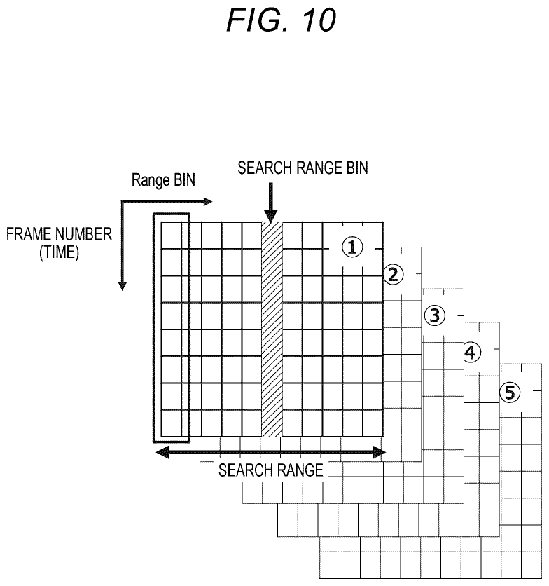

FIG. 10 is an image diagram of a waveform buffer according to the present embodiment. In FIG. 10, squares on a horizontal axis of a matrix indicate search range bins, and squares on a vertical axis of a matrix indicate one frame of the IQ data. A width of the horizontal axis of the matrix indicates a length of the search range, and a length of the vertical axis of the matrix indicates the length of the unit time for which the breathing rate is estimated. Selecting the search range bin in step S208 corresponds to selecting one of the squares on the horizontal axis of the matrix.

When the IQ data of one frame is buffered as needed and the IQ data is buffered by the length of the vertical axis, the subsequent IQ data is buffered in the next matrix. Thus, the breathing rate in each search range bin can be estimated in units of matrix. In addition, it is possible to estimate the breathing rate with higher accuracy by statistically processing the breathing rate estimated from each of the plurality of matrices.

(S213) The displacement amount calculation unit 410 calculates a displacement amount from the intensity waveform or the phase waveform buffered in the waveform buffering unit 409. This displacement amount corresponds to a displacement amount of the body surface due to the breathing of the person. The displacement amount calculation unit 410 may use the magnitude of the amplitude of the intensity waveform or the phase waveform as the displacement amount. Alternatively, the displacement amount calculation unit 410 may use the magnitude of the variance of the intensity waveform or the phase waveform as the displacement amount.

(S214) The breathing arrest determination unit 411 determines whether breathing has stopped based on a displacement amount calculated in step S213. For example, the breathing arrest determination unit 411 determines that breathing has stopped when the displacement amount is less than a predetermined threshold value, and determines that breathing has not stopped when the displacement amount is equal to or larger than the predetermined threshold value. At this time, since there is a difference in the magnitude of the detectable displacement amount depending on the detection position or the position of the body surface, the threshold value can be adaptively changed. The threshold value is determined based on a displacement amount in a predetermined period after the person stays at the position and a stillness determination result becomes valid. A value obtained by multiplying the threshold value determined based on the displacement amount during a predetermined period by a predetermined ratio may be used as the threshold value. The predetermined period and the predetermined ratio may be set and changed by the user. For example, the breathing arrest determination unit 411 determines the threshold value when the displacement amount during the predetermined period is relatively great to be a value larger than the threshold value when the displacement amount during the predetermined period is relatively small. Accordingly, since the threshold value is adaptively changed according to the magnitude of the displacement amount, the breathing arrest determination unit 411 can appropriately determine whether the breathing has stopped regardless of the detection position or the position of the body surface by using the threshold value.

(S215) The respiratory waveform filter unit 412 applies a predetermined respiratory waveform filter to the phase waveform buffered in the waveform buffering unit 409. The respiratory waveform filter may be a bandpass filter that extracts a component in a predetermined frequency range from the phase waveform. The predetermined frequency range may be determined by a general breathing rate of a human. Hereinafter, a phase waveform that does not pass through the respiratory waveform filter unit 412 is referred to as a first phase waveform, and a phase waveform that passes through the respiratory waveform filter unit 412 is referred to as a second phase waveform.

(S216) The unwrapping processing unit 413 performs unwrapping processing on each of the first phase waveform and the second phase waveform. When the displacement amount of the body surface due to breathing is larger than the wavelength of the radar, the phase waveform is folded back by one or more rotations of the phase. The unwrapping processing is processing of unwrapping the phase waveform. Thus, the phase waveform becomes a waveform representing the displacement amount and a displacement period of the body surface. The phase waveform used in the processing of the following step S217 to step S219 is the phase waveform after the unwrapping processing.

(S217) The frequency analysis unit 414 performs frequency analysis on the first phase waveform to calculate a first phase waveform spectrum, and performs frequency analysis on the second phase waveform to calculate a second phase waveform spectrum. The frequency conversion may be performed by, for example, FFT, short time fast Fourier transform (STFFT), or wavelet transform. In addition, the frequency analysis unit 414 may perform linear regression using the phase waveform spectrum as a log scale to specify and remove 1/f noise. Accordingly, the difference between the noise and the breathing spectrum becomes clearer.

(S218) The breathing rate estimation unit 415 estimates a first breathing rate from the first phase waveform spectrum and estimates a second breathing rate from the second phase waveform spectrum. Next, a method for estimating the breathing rate from the phase waveform spectrum will be described with reference to FIG. 11.

FIG. 11 is a diagram illustrating an example of a phase waveform spectrum according to the present embodiment.

The breathing rate estimation unit 415 detects the maximum peak of the spectrum in the frequency range corresponding to a breathing range of the person, and estimates a frequency bin of the maximum peak as the breathing rate of the person. The breathing range of a person may be set as an external parameter.

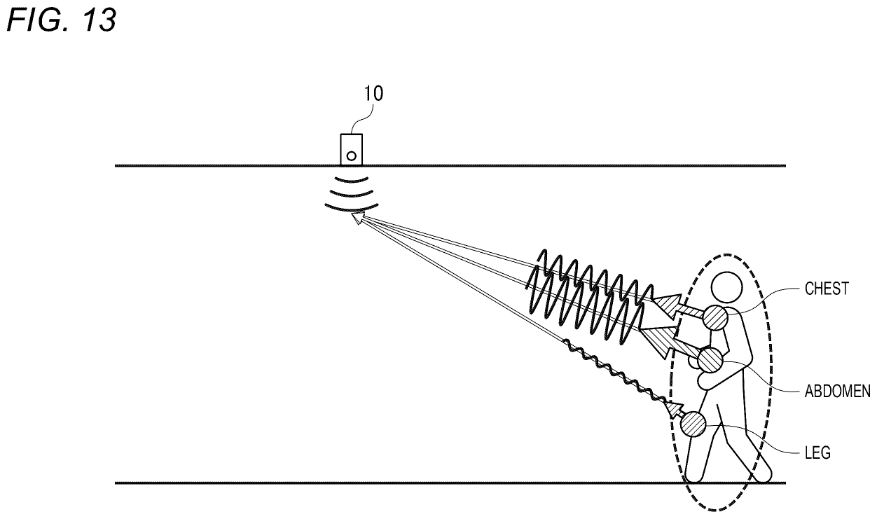

(S219) The likelihood calculation unit 416 calculates a first likelihood from the first phase waveform spectrum and calculates a second likelihood from the second phase waveform spectrum. The likelihood calculation unit 416 sets the larger of the first likelihood and the second likelihood as the likelihood of the search range bin selected in step S208, and sets the breathing rate having the larger likelihood as the breathing rate of the search range bin. Details of a likelihood calculation method will be described later (see FIGS. 13 to 16).

(S220) After performing the processing from step S208 to step S220 for all the search range bins, the breathing rate estimation processing unit 400 advances the processing to the next step S221. Through the processing from step S208 to step S220, the breathing rate and the likelihood for each search range bin are obtained.

(S221) The breathing rate selection unit 417 sets the breathing rate to 0) when the determination result by the breathing arrest determination unit 411 is breathing arrest. When the determination result by the breathing arrest determination unit 411 is not breathing arrest, the breathing rate selection unit 417 performs the following processing. That is, the breathing rate selection unit 417 selects the breathing rate of the search range bin having the highest likelihood among the plurality of search range bins as the breathing rate of the person. When there are a plurality of highest likelihoods, the breathing rate selection unit 417 may select the breathing rate of the range bin having the largest displacement amount as the breathing rate of the person.

(S222) The breathing rate filter unit 418 applies a smoothing filter to a time fluctuation of the breathing rate selected in step S221. Examples of the smoothing filter include a moving average filter, a median filter, and a Kalman filter. As a result, it is possible to prevent the breathing rate from fluctuating unnecessarily over time, for example, when the estimation accuracy of the breathing rate temporarily deteriorates.

(S224) After the breathing rate estimation processing unit 400 performs the processing from step S206 to step S223 for all the persons present in the room, the processing proceeds to step S224. Through the processing from step S206 to step S223, the breathing rate of each person present in the room is obtained.

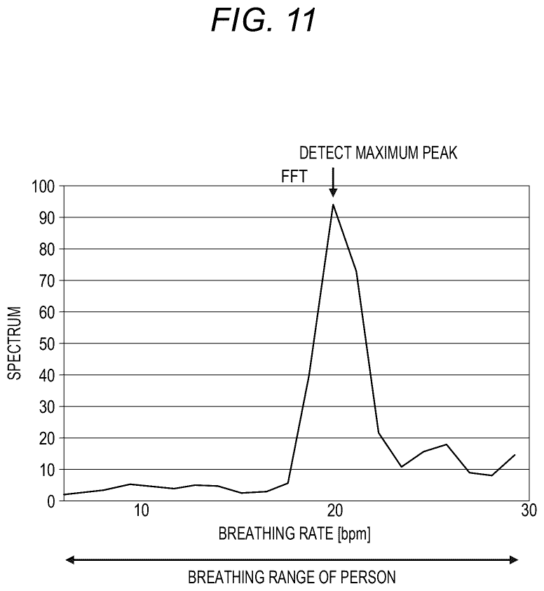

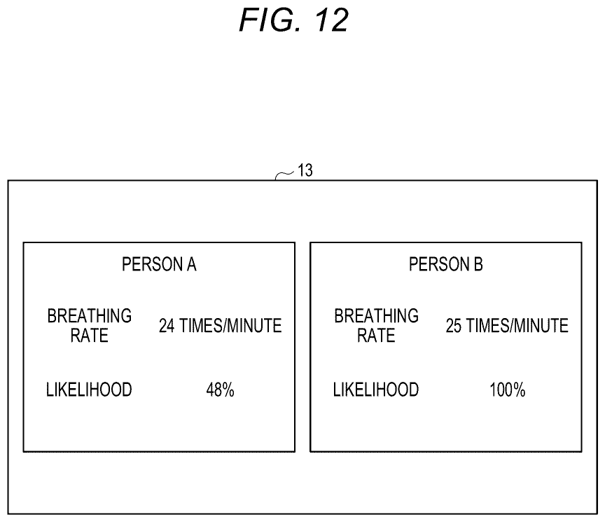

(S224) As illustrated in a display example of the breathing rate and the likelihood in FIG. 12, the browsing processing device 12 displays the breathing rate and the likelihood of each person obtained by the above processing on the display device 13. This allows the user to confirm the breathing rate and likelihood of each person present in the room.

### <Description of Likelihood>

Next, the likelihood will be described in detail.

FIG. 13 is a diagram illustrating an example of a reflection position of the radar with respect to the body surface of the person according to the present embodiment.

The radar device 10 receives the reflected waves arriving from different positions (that is, different search range bins) on the body surface of a person. Hereinafter, a method for calculating the likelihood will be described focusing on reflected waves arriving from the abdomen of the person, reflected waves arriving from the chest of the person, and reflected waves arriving from the leg of the person.

In this case, the breathing rate estimation processing unit 400 acquires IQ data of reflected waves arriving from the abdomen of the person (hereinafter referred to as abdomen IQ data), IQ data of the reflected waves arriving from the chest of the person (hereinafter referred to as chest IQ data), and IQ data of the reflected waves arriving from the leg of the person (hereinafter referred to as leg IQ data).

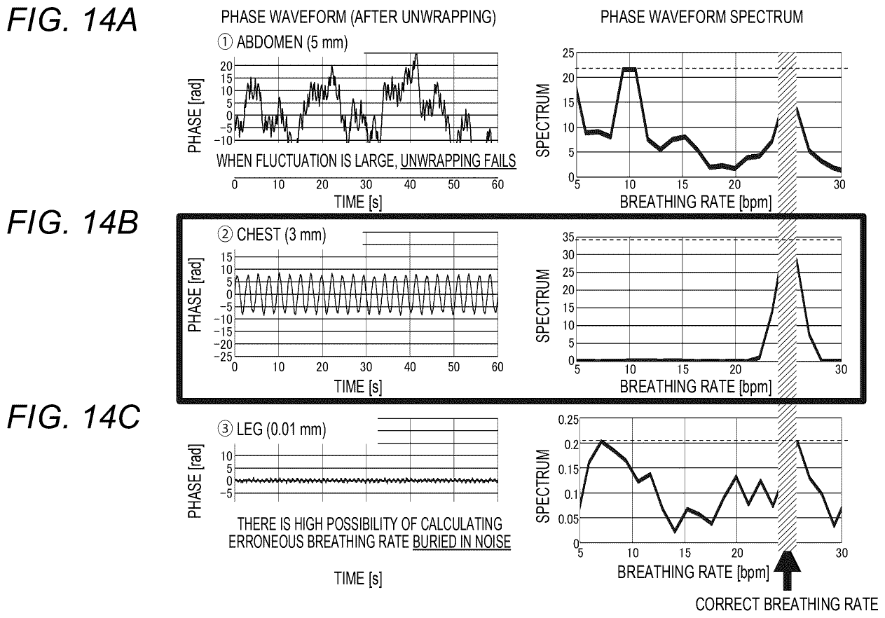

FIG. 14A to 14C are graphs illustrating phase waveforms and phase waveform spectra in for the abdomen, the chest, and the leg according to the present embodiment.

FIG. 14A illustrates a phase waveform in the abdomen (hereinafter referred to as an abdomen phase waveform) calculated based on the abdomen IQ data and a phase waveform spectrum obtained by frequency-converting the abdomen phase waveform (hereinafter referred to as an abdomen phase waveform spectrum).

FIG. 14B illustrates a phase waveform in the chest (hereinafter referred to as a chest phase waveform) calculated based on the chest IQ data and a phase waveform spectrum obtained by frequency-converting the chest phase waveform (hereinafter referred to as a chest phase waveform spectrum).

FIG. 14C illustrates a phase waveform in the leg (hereinafter referred to as a leg phase waveform) calculated based on the leg IQ data and a phase waveform spectrum obtained by frequency-converting the leg phase waveform (hereinafter referred to as a leg phase waveform spectrum).

When the fluctuation of the body surface is large, for example, 5 mm as in the abdomen, an abdomen phase waveform that has failed to be unwrapped can be calculated as illustrated in FIG. 14A. In this way, when the breathing rate is estimated from the abdomen phase waveform spectrum corresponding to the abdomen phase waveform that has failed to be unwrapped, an erroneous breathing rate is estimated. Therefore, the likelihood of the abdomen phase waveform spectrum as illustrated in FIG. 14A should be calculated to be small.

When the fluctuation of the body surface is appropriate, for example, 3 mm as in the chest, as illustrated in FIG. 14B, a chest phase waveform that has been successfully unwrapped and has a sufficiently high signal noise (SN) ratio can be calculated. In this way, the correct breathing rate can be estimated by estimating the breathing rate from the chest phase waveform spectrum corresponding to the chest phase waveform that has been successfully unwrapped and has the sufficiently high SN ratio. Therefore, the likelihood of the chest phase waveform spectrum as illustrated in FIG. 14B should be calculated to be high.

When the fluctuation of the body surface is as small as, for example, 0.01 mm as in the leg, a leg phase waveform having an insufficient SN ratio (low SN ratio) can be calculated as illustrated in FIG. 14C. As described above, when the breathing rate is estimated from the leg phase waveform spectrum corresponding to the leg phase waveform having the insufficient SN ratio, an erroneous breathing rate is estimated. Therefore, the likelihood of the leg phase waveform spectrum as illustrated in FIG. 14C should be calculated to be small.

The likelihood calculation unit 416 calculates a high likelihood for a phase waveform spectrum with a low possibility that an erroneous breathing rate is estimated, and calculates a low likelihood for a phase waveform spectrum with a high possibility that an erroneous breathing rate is estimated. Accordingly, the breathing rate selection unit 417 can select a more correct breathing rate without selecting an erroneous breathing rate among the breathing rates estimated for different positions (that is, different search range bins) on the body surface of the person with reference to the likelihood.

Next, a method for calculating the likelihood from the phase waveform spectrum will be described.

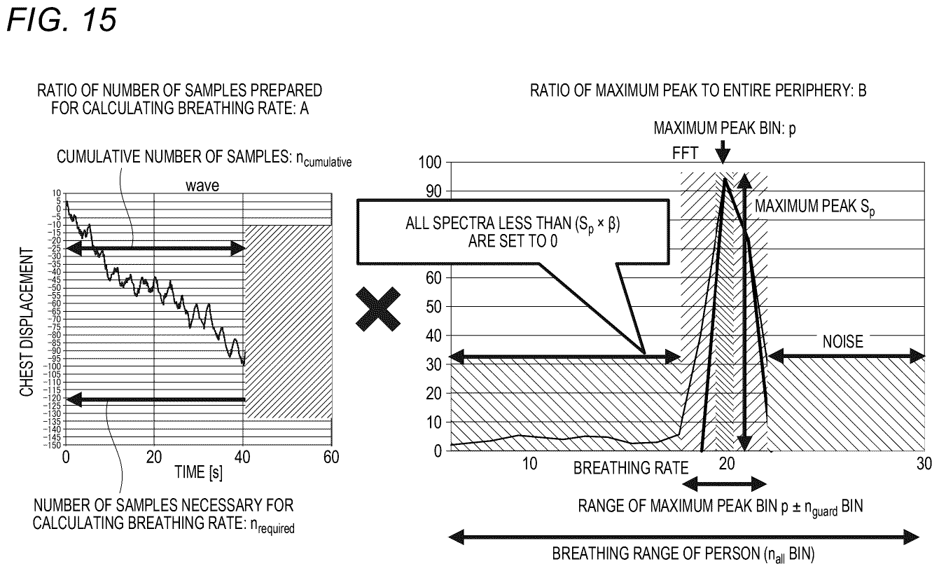

FIG. 15 is a diagram illustrating a method for calculating the likelihood according to the present embodiment.

First, the likelihood calculation unit 416 calculates a ratio A of samples prepared for estimating the breathing rate by the following equation (1).

$$A = {n}_{cumulative} / {n}_{required} \qquad ( 1 )$$

Here, n_{required} represents the number of samples required to estimate the breathing rate per unit time (for example, one minute), and n_{cumulative} represents the cumulative number of samples. The number of samples required to estimate the breathing rate per unit time may be a phase waveform per unit time.

Next, the likelihood calculation unit 416 calculates a ratio B of the maximum peak of the phase waveform spectrum and a periphery thereof to the whole. This will be described in detail below.

First, the likelihood calculation unit 416 specifies a frequency bin (hereinafter, referred to as a maximum peak bin) p of the maximum peak Sₚ having the maximum spectrum in the phase waveform spectrum.

Next, the likelihood calculation unit 416 calculates the maximum peak Sₚ×β. β is a predetermined value of 0<β<1. That is, the likelihood calculation unit 416 calculates a predetermined ratio (for example, 10%) of the maximum peak Sₚ.

Next, the likelihood calculation unit 416 sets all spectra less than “Sₚ×β” to 0 in a breathing frequency spectrum, and generates the adjusted phase waveform spectrum Si.

Next, the likelihood calculation unit 416 calculates the following equation (2) for the adjusted phase waveform spectrum Si.

$$B = {S}_{p g} / ( {S}_{p g} + N ) \qquad ( 2 )$$

Here, S_{pg} is calculated by the following equation (3). (S_{pg}+N) is calculated by the following equation (4). n_{guard} is a preset value. N indicates a spectrum of a frequency bin other than the frequency bins between p−n_{guard} and p+n_{guard} among all the frequency bins nan in the breathing range of a person.

$$\begin{matrix}[ Math . 1 ] & \\ {S}_{p g} = {\sum }_{i = p - {n}_{g u a r d}}^{p + {n}_{guard}} {S}_{i} & ( 3 )\end{matrix}$$

$$\begin{matrix}[ Math . 2 ] & \\ {S}_{p g} + N = {\sum }_{{i = 1}^{}}^{{n}_{a l l}} {S}_{i} & ( 4 )\end{matrix}$$

That is, B indicates the ratio of the cumulative total (S_{pg}) of the spectra in a predetermined range including the maximum peak to the cumulative total (S_{pg}+N) of the spectra that are not 0 in the frequency bins nan in the breathing range of the person. B may be read as a characteristic of the fluctuation or a ratio of a peak spectrum to noise in the breathing range of the person.

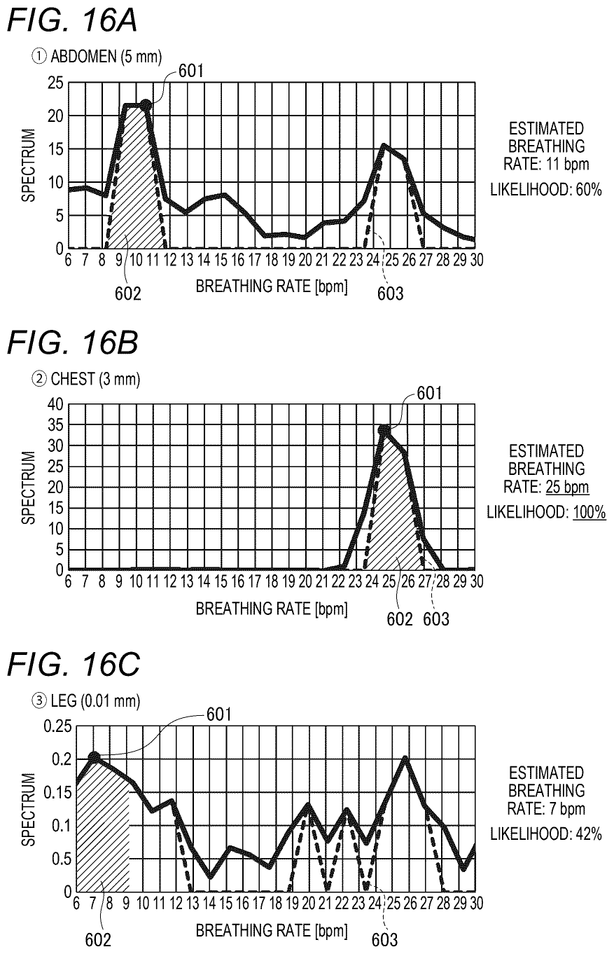

The likelihood calculation unit 416 calculates A x B as the likelihood. Note that the likelihood calculation unit 416 may calculate only B as the likelihood without using A. FIGS. 16A to 16C are a diagram illustrating an example of the phase waveform

spectra and likelihoods of the abdomen, the chest, and the leg according to the present embodiment.

In FIGS. 16A to 16C, a circular point 601 indicates the maximum peak Sp, a hatched area 602 indicates (S_{pg}), and a dotted area indicates (S_{pg}+N).

As illustrated in FIG. 16A, the breathing rate estimation unit 415 estimates the breathing rate as 11 breath per minutes (bpm) from the abdomen phase waveform spectrum as indicated by the maximum peak bin p of the maximum peak Sₚ at the round point 601. The likelihood calculation unit 416 calculates the likelihood as 60% from the abdomen phase waveform spectrum as indicated by the ratio of the hatched area 602 to the dotted area 603.

As illustrated in FIG. 16B, the breathing rate estimation unit 415 estimates the breathing rate as 25 bpm from the chest phase waveform spectrum as indicated by the maximum peak bin p of the maximum peak Sₚ at the round point 601. The likelihood calculation unit 416 calculates the likelihood as 100% from the chest phase waveform spectrum as indicated by the ratio of the hatched area 602 to the dotted area 603.

As illustrated in FIG. 16C, the breathing rate estimation unit 415 estimates the breathing rate as 7 bpm from the leg phase waveform spectrum as indicated by the maximum peak bin p of the maximum peak Sₚ at the round point 601. The likelihood calculation unit 416 calculates the likelihood as 42% from the leg phase waveform spectrum as indicated by the ratio of the hatched area 602 to the dotted area 603.

In this case, since the likelihood of the chest phase waveform spectrum is the highest, the breathing rate selection unit 417 selects the breathing rate of 25 bpm for the chest phase waveform spectrum. Accordingly, the breathing rate estimation system 1 can output the correct breathing rate as much as possible estimated at an appropriate position on the body surface of the person. In other words, it is possible to prevent the breathing rate estimation system 1 from outputting an erroneous breathing rate estimated at an inappropriate position on the body surface of the person.

### (Hardware Configuration)

The functional blocks of the breathing rate estimation device 11 described above can be realized by a computer program.

FIG. 17 is a diagram illustrating a hardware configuration example of an information processing device (computer) that realizes functional blocks of the breathing rate estimation device 11 according to the present disclosure by a computer program.

The information processing device 1000 includes a processor 1001, a memory 1002, a storage 1003, an input interface (I/F) 1004, an output I/F 1005, a communication I/F 1006, a graphics processing unit (GPU) 1007, a reading I/F 1008, and a bus 1009.

The processor 1001, the memory 1002, the storage 1003, the input I/F 1004, the output I/F 1005, the communication I/F 1006, the graphics processing unit (GPU) 1007, and the reading I/F 1008 are connected to the bus 1009 and can bidirectionally transmit and receive data via the bus 1009.

The processor 1001 is a device that executes a computer program stored in the memory 1002 to implement the functional blocks described above. Examples of the processor 1001 include a central processing unit (CPU), a micro processing unit (MPU), a controller, a large scale integration (LSI), an application specific integrated circuit (ASIC), a programmable logic device (PLD), and a field-programmable gate array (FPGA).

The memory 1002 is a device that stores a computer program and data handled by the information processing device 1000. The memory 1002 may include a read-only memory (ROM) and a random access memory (RAM).

The storage 1003 is a device that is implemented by a nonvolatile storage medium, and that stores a computer program and data handled by the information processing device 1000. Examples of the storage 1003 include a hard disk drive (HDD) and a solid state drive (SSD).

The input I/F 1004 is connected to an input device that receives an input from a user, and transmits data received from the input device to the processor 1001. Examples of the input device include a keyboard, a mouse, a touch pad, and a microphone.

The output I/F 1005 is connected to an output device and transmits data received from the processor 1001 to the output device. Examples of the output device include a display device and a speaker.

The communication I/F 1006 is connected to the communication network and transmits and receives data to and from another device (for example, the browsing processing device 12) via the communication network. The communication I/F 1006 may support either wired communication or wireless communication. Examples of the wired communication include Ethernet (registered trademark). Examples of the wireless communication include Wi-Fi (registered trademark), Bluetooth (registered trademark), long term evolution (LTE), 4G, and 5G.

The GPU 1007 is a device that processes image depiction at a high speed. The GPU 1007 may be used for processing (for example, deep learning processing) of artificial intelligence (AI).

The reading I/F 1008 is connected to an external storage medium and reads data from the external storage medium. Examples of the external storage medium include a digital versatile disk read only memory (DVD-ROM) and a universal serial bus (USB) memory:

The functional blocks of the breathing rate estimation device 11 may be implemented as an LSI that is an integrated circuit. These functional blocks may be individually integrated into one chip, or may include some or all of these functions into one chip. Here, the function is implemented as an LSI. Alternatively, the function may also be called an IC, a system LSI, a super LSI, or an ultra LSI depending on the degree of integration. Further, if an integrated circuit technique that replaces the LSI emerges due to an advancement in semiconductor technique or another derived technique, the functional blocks may naturally be integrated using that technique.

### Summary of Present Disclosure

The following techniques are disclosed based on the above description of the embodiment.

### Technique 1

The breathing rate estimation system 1 includes at least one radar type sensor (for example, the radar device 10) installed in the monitoring area, an information processing device (for example, the breathing rate estimation device 11) configured to execute the person detection processing (for example, the person detection processing unit 300) of acquiring sensor data output from the sensor and detecting a person present in the monitoring area using the acquired sensor data and the breathing rate estimation processing (for example, the breathing rate estimation processing unit 400) of estimating the breathing rate of the person, and the display device 13 configured to display the breathing rate of the person estimated in the breathing rate estimation processing. The information processing device determines whether the person is in the specific state based on the detection result of the person by the person detection processing, and executes the breathing rate estimation processing when it is determined that the person is in the specific state.

Accordingly, since the information processing device executes the breathing rate estimation processing when the person is in the specific state and displays the estimated breathing rate of the person, it is possible to prevent an erroneous breathing rate from being estimated and displayed when the person is not in the specific state.

### Technique 2

In the breathing rate estimation system 1 according to Technique 1, in the person detection processing, a determination is made as to whether the person is in the static state as the determination of whether the person is in the specific state.

Accordingly, since the information processing device executes the breathing rate estimation processing when the person is in the static state and displays the estimated breathing rate of the person, it is possible to prevent an erroneous breathing rate from being estimated and displayed when the person is not in the static state.

### Technique 3

In the breathing rate estimation system 1 according to Technique 1 or 2, in the breathing rate estimation processing, fluctuations at different positions on the body surface of the person are calculated using the sensor data, the breathing rate corresponding to each position is estimated based on the calculated fluctuation, and the likelihood of the breathing rate is calculated.

Accordingly, the certainty of the breathing rate corresponding to each position can be recognized by the likelihood associated with the breathing rate.

### Technique 4

In the breathing rate estimation system 1 according to Technique 3, in the breathing rate estimation processing, the likelihood is calculated in accordance with a ratio of accumulation per unit time of the data indicating the fluctuation and characteristic of the fluctuation.

Thus, the likelihood of the breathing rate can be calculated.

### Technique 5

In the breathing rate estimation system 1 according to Technique 3 or 4, in the breathing rate estimation processing, the breathing rate having the largest likelihood among the likelihoods corresponding to the respective positions is estimated as the breathing rate of the person.

Accordingly, the breathing rate of the person can be estimated with higher accuracy.

### Technique 6

In the breathing rate estimation system 1 according to any one of Techniques 1 to 5, in the breathing rate estimation processing, it is determined whether the person is in a breathing arrest state based on the fluctuation, and the breathing rate of the person is not estimated when it is determined that the person is in the breathing arrest state.

Accordingly, it is possible to prevent an erroneous breathing rate from being estimated and displayed when the person is in the breathing arrest state.

### Technique 7

In the breathing rate estimation system 1 according to Technique 6, in the breathing rate estimation processing, a threshold value is determined based on the magnitude of the fluctuation during a predetermined period after it is determined that the person is in the static state, and it is determined whether the person is in the breathing arrest state based on the threshold value and the fluctuation.

Accordingly, since the threshold value is adaptively changed in accordance with the magnitude of the fluctuation, it is possible to appropriately determine whether the breathing has stopped regardless of the detection position or the position of the body surface by using the threshold value in the breathing rate estimation processing.

### Technique 8

In the breathing rate estimation system 1 according to Technique 5, the information processing device displays the breathing rate of the person and the likelihood of the breathing rate in association with each other on the display device 13.

This allows the user to know the likelihood (certainty) of the breathing rate in addition to the breathing rate of the person.

### Technique 9

In the breathing rate estimation system 1 according to any one of Techniques 1 to 8, the sensor data includes point cloud data indicating the position of the person and IQ data indicating the fluctuation of the body surface of the person, in the person detection processing, the person is detected using the point cloud data, and in the breathing rate estimation processing, the breathing rate of the person is estimated using the IQ data corresponding to the position of the person detected in the person detection processing.

In this way, by estimating the breathing rate of the person using the IQ data corresponding to the position of the person detected using the point cloud data, the breathing rate of the person can be estimated with higher accuracy.

### Technique 10

The breathing rate estimation device 11 includes the processor 1001 and the memory 1002, and the processor 1001 cooperates with the memory 1002 to acquire sensor data output from at least one radar type sensor (for example, the radar device 10) installed in the monitoring area, execute person detection processing (for example, the person detection processing unit 300) of detecting a person present in the monitoring area using the sensor data, determine whether the person is in a specific state based on a detection result of the person in the person detection processing, execute breathing rate estimation processing (for example, the breathing rate estimation processing unit 400) of estimating the breathing rate of the person using the sensor data when it is determined that the person is in the specific state, and output the breathing rate of the person estimated in the breathing rate estimation processing.

Accordingly, the breathing rate estimation device 11 executes the breathing rate estimation processing when the person is in the specific state, and displays the estimated breathing rate of the person, and thus it is possible to prevent an erroneous breathing rate from being estimated and displayed when the person is not in the specific state.

### Technique 11

A breathing rate estimation method includes: acquiring sensor data output from at least one radar type sensor (for example, the radar device 10) installed in the monitoring area:

executing person detection processing (for example, the person detection processing unit 300) of detecting a person present in the monitoring area using the sensor data: determining whether the person is in a specific state based on a detection result of the person in the person detection processing: executing breathing rate estimation processing (for example, the breathing rate estimation processing unit 400) of estimating a breathing rate of the person using the sensor data when it is determined that the person is in the specific state; and outputting the breathing rate of the person estimated in the breathing rate estimation processing.

As described above, since the breathing rate estimation processing is executed when the person is in the specific state and the estimated breathing rate of the person is displayed in the breathing rate estimation method, it is possible to prevent an erroneous breathing rate from being estimated and displayed when the person is not in the specific state.

### Technique 12

A breathing rate estimation program causes a computer to execute processing, the processing including: acquiring sensor data output from at least one radar type sensor (for example, the radar device 10) installed in the monitoring area: executing person detection processing (for example, the person detection processing unit 300) of detecting a person present in the monitoring area using the sensor data: determining whether the person is in a specific state based on a detection result of the person in the person detection processing: executing breathing rate estimation processing (for example, the breathing rate estimation processing unit 400) of estimating a breathing rate of the person using the sensor data when it is determined 30) that the person is in the specific state; and outputting the breathing rate of the person estimated in the breathing rate estimation processing.

As described above, since the breathing rate estimation program executes the breathing rate estimation processing when the person is in the specific state and displays the estimated breathing rate of the person, it is possible to prevent an erroneous breathing rate from being estimated and displayed when the person is not in the specific state.

Although the embodiment has been described above with reference to the accompanying drawings, the present disclosure is not limited thereto. It is apparent to those skilled in the art that various modifications, corrections, substitutions, additions, deletions, and equivalents can be conceived within the scope described in the claims, and it is understood that such modifications, corrections, substitutions, additions, deletions, and equivalents also fall within the technical scope of the present disclosure. In addition, constituent elements in the embodiment described above may be freely combined without departing from the gist of the invention.

The present application is based on a Japanese Patent Application (Japanese Patent Application No. 2022-144863) filed on Sep. 12, 2022, and the contents thereof are incorporated herein by reference.

### INDUSTRIAL APPLICABILITY

The techniques of the present disclosure are useful for estimating the breathing rate of a person.

### REFERENCE SIGNS LIST

1 breathing rate estimation system
10 radar device
11 breathing rate estimation device
12 browsing processing device
13 display device
21 signal generator
22 amplifier
23 transmission antenna
24 reception antenna
25 noise reducer
26 mixer
27 AD converter
28 signal processor
29 processor
300 person detection processing unit
301 point cloud data acquisition unit
302 clustering unit
303 tracking unit
304 stillness determination unit
305 person information output unit
400 breathing rate estimation processing unit
401 IQ data acquisition unit
402 Range FFT unit
403 IQ correction unit
404 person information acquisition unit
405 coordinate conversion unit
406 mode vector multiplication unit
407 intensity extraction unit
408 phase extraction unit
409 waveform buffering unit
410 displacement amount calculation unit
411 breathing arrest determination unit
412 respiratory waveform filter unit
413 unwrapping processing unit
414 frequency analysis unit
415 breathing rate estimation unit
416 likelihood calculation unit
417 breathing rate selection unit
418 breathing rate filter unit

1. A breathing rate estimation system comprising:
at least one radar type sensor installed in a monitoring area:
an information processing device that executes person detection processing of acquiring sensor data output from the sensor and detecting a person present in the monitoring area using the acquired sensor data and breathing rate estimation processing of estimating a breathing rate of the person; and
a display device that displays the breathing rate of the person estimated in the breathing rate estimation processing, wherein
the information processing device determines whether the person is in a specific state based on a detection result of the person by the person detection processing, and executes the breathing rate estimation processing when it is determined that the person is in the specific state.

2. The breathing rate estimation system according to claim 1, wherein
in the person detection processing, a determination is performed as to whether the person is in a still state as the determination of whether the person is in the specific state.

3. The breathing rate estimation system according to claim 2, wherein
in the breathing rate estimation processing,
fluctuations at different positions on a body surface of the person are calculated using the sensor data, and
the breathing rate corresponding to the respective positions is estimated based on the calculated fluctuation, and a likelihood of the breathing rate is calculated.

4. The breathing rate estimation system according to claim 3, wherein
in the breathing rate estimation processing, the likelihood is calculated in accordance with a ratio of accumulation per unit time of data indicating the fluctuation and characteristic of the fluctuation.

5. The breathing rate estimation system according to claim 3, wherein
in the breathing rate estimation processing, the breathing rate having a highest likelihood among the likelihoods corresponding to the respective positions is estimated as the breathing rate of the person.

6. The breathing rate estimation system according to claim 5, wherein
in the breathing rate estimation processing, it is determined whether the person is in a breathing arrest state based on the fluctuation, and
when it is determined that the person is in the breathing arrest state, the breathing rate of the person is not estimated.

7. The breathing rate estimation system according to claim 6, wherein
in the breathing rate estimation processing, a threshold value is determined based on a magnitude of the fluctuation during a predetermined period after it is determined that the person is in the static state, and it is determined whether the person is in the breathing arrest state based on the threshold value and the fluctuation.

8. The breathing rate estimation system according to claim 5, wherein
the information processing device displays the breathing rate of the person and the likelihood of the breathing rate in association with each other on the display device.

9. The breathing rate estimation system according to any one of claims 1 to 8, wherein the sensor data includes point cloud data indicating a position of the person and IQ data indicating the fluctuation of the body surface of the person,
in the person detection processing, the person is detected using the point cloud data, and
in the breathing rate estimation processing, the breathing rate of the person is estimated using the IQ data corresponding to the position of the person detected in the person detection processing.

10. A breathing rate estimation device comprising:
a processor; and
a memory, wherein
the processor cooperates with the memory to
acquire sensor data output from at least one radar type sensor installed in a monitoring area,
execute person detection processing of detecting a person present in the monitoring area using the sensor data,
determine whether the person is in a specific state based on a detection result of the person in the person detection processing,
execute breathing rate estimation processing of estimating a breathing rate of the person using the sensor data when it is determined that the person is in the specific state, and
output the breathing rate of the person estimated in the breathing rate estimation processing.

11. A breathing rate estimation method comprising:
acquiring sensor data output from at least one radar type sensor installed in a monitoring area:
executing person detection processing of detecting a person present in the monitoring area using the sensor data:
determining whether the person is in a specific state based on a detection result of the person in the person detection processing:
executing breathing rate estimation processing of estimating a breathing rate of the person using the sensor data when it is determined that the person is in the specific state; and
outputting the breathing rate of the person estimated in the breathing rate estimation processing.

12. A breathing rate estimation program causing a computer to execute processing, the processing including:
acquiring sensor data output from at least one radar type sensor installed in a monitoring area:
executing person detection processing of detecting a person present in the monitoring area using the sensor data:
determining whether the person is in a specific state based on a detection result of the person in the person detection processing:
executing breathing rate estimation processing of estimating a breathing rate of the person using the sensor data when it is determined that the person is in the specific state; and
outputting the breathing rate of the person estimated in the breathing rate estimation processing.
